(19) United States
(12) Patent Application Publication
(10) Pub. No.: US 2024/0198053 A1
(43) Pub. Date: Jun. 20, 2024

(54) MEDICAL DEVICE FOR SELF-CATHETERIZATION AND/OR INSTILLATION OF MEDICAMENTS INTO BLADDER OF FEMALE PATIENTS

(71) Applicants: Sándor LOVÁSZ, Budapest (HU); Gábor RÉNYI, Budapest (HU); János GIBER, Budapest (HU)

(72) Inventors: Sándor LOVÁSZ, Budapest (HU); Gábor RÉNYI, Budapest (HU); János GIBER, Budapest (HU)

(21) Appl. No.: 18/593,202

(22) Filed: Mar. 1, 2024

Related U.S. Application Data
(63) Continuation-in-part of application No. 16/074,640, filed on Aug. 1, 2018.

(30) Foreign Application Priority Data
Feb. 1, 2016 (HU) P1600050

(51) Int. Cl. A61M 25/01; A61M 39/22

(52) U.S. Cl. CPC A61M 25/0136; A61M 39/223; A61M 2202/0496; A61M 2205/3306; A61M 2205/583; A61M 2205/587

(57) ABSTRACT

A medical device suitable for self-catheterization by a female patient includes a female catheter associated with a three-way valve provided with a rigid grip which carries a visual aid.

### CROSS-REFERENCE TO RELATED APPLICATION

This application is a continuation-in-part of U.S. Ser. No. 16/074,640, filed Aug. 1, 2018, which in turn is a U.S. National Stage application under 35 U.S.C. 371 of PCT/HU2017/000006, filed Feb. 1, 2017; and claims priority of Hungarian Patent Application P1600050, filed Feb. 1, 2016, each of which is incorporated herein by reference in entirety.

### FIELD OF INVENTION

This invention relates to medical devices for self-catheterization and instillation of medication in female patients.

### BACKGROUND OF THE INVENTION

Thousands of female patients have urinary retention or problems with bladder drainage because of nerve injury to the spinal cord or after difficult urination the high pressure and quantity of the residual urine are endangering the kidney function and/or causing an intractable urinary tract infection.

Multiple nerve injuries generated during radical pelvic surgeries used because of the cancer processes of the pelvic tract (colon cancer, uterine tumors, ovarian cancer) is also another frequent reason for treatment of ischuria.

In such cases previously, patients were wearing a so called “permanent” balloon-catheter for a whole lifetime with surgical replacements every 2-6 weeks, depending on the material of the catheter.

For this traditional solution, the patients are transported to the surgeries or treated at home, and this is an enormous healthcare burden.

Wearing a permanent catheter causes continuous irritation, pain, bleeding in urine, and deterioration in the quality of life.

Serious complications such as chronic bacterial urinary tract infections and resulting inflammation of the renal pelvis, or purulent inflammation of urethra with high fever could adversely affect the life expectancy of patients.

In recent decades, a professionally increasingly recommended method is known, the so called Intermittent Clean Self-Catheterization performed by the patients on their own.

The insertion of the catheter into the external urethral orifice (urinary meatus, meatal opening) and the drainage of the bladder is performed mostly by the patient sometimes using external assistance.

There are many difficulties and disadvantages in clean self-catheterization of female patients. For exploration of the external urethral orifice and the insertion of the catheter, a high degree of manual dexterity and motility is required by the female patients.

Many patients are not capable of self-catheterization on their own, only by using external assistance. Maintaining proper sterility is very difficult, almost impossible, using the method of permanent self-catheterization in its present form.

For a significant portion of the previous type of patients, regular local medication of the bladder is needed to treat chronic bacterial inflammation, bleeding, or other associative bladder diseases.

By using the known, traditional self-catheterization method, these kinds of treatments are hard to carry out, because the syringe (20 cm) connecting to the drain-catheter (20-30 cm) is practically in unattainable distance from the patient.

For another type of patient, no bladder drainage is needed because they are capable of spontaneous drainage. However, because of their basic diseases, regular local medication of the bladder is needed.

The basic diseases are the complications following radiotherapy of cancer of the pelvic tract (radiation injury and tissue damage of a part of the bladder, mostly the base of the bladder or the urethra with pain, bleeding, and problems with micturition.

The most effective treatment of these diseases is the instillation of bladder with drug solutions (GAG layer remounts and regenerators) and used by the medication of Bladder Pain Syndrome/Interstitialis Cystitis (BPS/IC).

Serious cases, such as injury from palliative radiation without hope of full recovery are included in this type of patient. During pelvic radiations concerning other indications, adjuvant or palliative treatments (in the case of advanced diseases) after surgical removal of bladder, colon, uterine and ovarian tumors, some parts of the bladder could be significantly radiated, therefore intravesical bladder instillation is needed.

BPS/IC patients living far from a medical assistance cannot be easily treated regularly because of the long journeys. Clean self-catheterization in this type of patient has the advantage that, the symptoms could be treated immediately with bladder instillations in the worse stages of the disease, there is no need to wait for a medication by the doctor.

The number of female BPS/IC patients is about 20,000, although the number of diagnosed cases are only fraction of the real value because of the bad discovery rate of the disease BPS/IC.

In regard to the fact that, the professional interest to the BPS/IC is growing worldwide more and more, the detection rate of the BPS/IC and the number of BPS/IC patients will also skyrocket.

BPS/IC patients can be treated in about 100% by a catheter-free bladder instillation by using a 16 syringe adapter for injection, protected by a patent application (Hungarian application number P1500648).

Chronic bacterial bladder inflammations existing for years can be detected more and more often caused by the pathogens being multi-resistant against all antibiotics. In such cases, the regeneration of the defending mucous-layer (glycosaminoglycan, the GAG layer) on the surface of the inner wall of the bladder by regular local bladder instillations is almost the only effective treatment.

Benefits of clean self-catheterization could be enjoyed by all types of patients if bladder instillation could be performed by their own. The difficulties and disadvantages of catheter-free self-bladder instillation are the same as for clean self-catheterization disclosed above.

Therefore, the medical device according to our invention consists mostly of the same elements, only some parts are different according to the special needs of different types of patients.

For self-catheterization in female patients, many solutions could be found previously. A device for self-catheterization in female patients with a tripod is disclosed by the patent application US 20150062729. The tripod is equipped with a movable mirror equipped with a light source with battery. The mirror is adjustable in different planes. With the assistance of the mirror and the light source, the device can be adjusted to the direction of the urethra. With the assistance of the device the urinary meatus can be seen by the women, assisting the self-catheterization in this way. A large-scale, complicated device is disclosed in this invention where either the installation, handling, or storage of the device is complicated and cumbersome. In addition, correct positioning of the patient and the device is cumbersome.

A device for self-catheterization for women in case of not proper bladder drainage is disclosed in U.S. Pat. No. 5,084,036 patent. The device consists of a basic part for the insertion into the vagina, of a support part being perpendicular to the basic part, which can be found on the opposite of the outer part of the vagina, and of a grip element which is part of the basic part. A transfer tube is connected to the basic part: one end of the tube is in front of the outer part of the vaginal opening (urinary meatus), and the other end of the tube can be connected to the end of the catheter for insertion, directed into the urethra, making the self-catheterization easier. Similar to the previous solution, the patented device is also large-scaled, complicated, difficult to handle, and storage and installation are cumbersome.

A device for insertion of the catheter and self-catheterization in female patients is disclosed in the patent document DE 10060088 consisting of a holder part for insertion into the vaginal opening and of a front leading part for the catheter. The front leading part and the holder part are placed at a distance appropriate to the distance between the vagina and the urethra with a spacer. The holder part is cone-shaped and its intersection is increasing while moving away from the spacer. The front leading part is releasable connected to the spacer and its intersection gets narrower in the direction to the spacer. This solution is very simple, but it is not suitable for ensuring attainment of our objective. The common disadvantage of the earlier known solutions of the prior art is that all subject matter of the inventions can only solve the problems of urine drainage with the earlier disclosed handicaps.

The objective of the present invention is to develop a device embodying the present invention which can be used by all types of patients. Handling the medical device is easy. It is simple for a patient to use.

### SUMMARY OF THE INVENTION

A medical device suitable for self-catheterization by a female patient is provided with a female catheter, a three-way valve operably associated with the catheter, a rigid grip attached to the three-way valve, and at least one visual aid mounted to the rigid grip. The visual aid can be fixed to the grip or removably attached thereto. The visual aid can be a mirror, a camera, and the like. Optionally, a light source can be mounted to the grip as well.

The medical device provides a solution for treatment and nursing of patients suffering from chronic diseases or living with disabilities, significantly reducing the number of complications of catheterization and the burden of catheterization on such patients and improving their life expectancy. Self-catheterization can be readily achieved.

The medical device can be installed under visual control according to the needs of all types of female patients. Using this medical device, the catheter can be inserted into the urethra using only one hand. The vulva can be explored properly, the urinary meatus can be located and catheterized easily. Therefore, a higher level of sterility can be assured.

The grip can be a syringe, a handle, and the like, and the catheter and a urine drainage bag can be connected through the three-way valve.

In use, the grip connected to the catheter is taken by one hand, the leading end of the catheter is placed near to the urinary meatus and the surrounding region illuminated. Next, the mirror and/or the camera are positioned so that the view shows the end of the catheter and the urinary meatus. Thus, the catheter can be inserted easily into the urinary meatus in a proper manner, and the urine drainage and/or the medicament instillation can be performed single-handedly by the patient herself.

### BRIEF DESCRIPTION OF THE DRAWINGS

In the drawings,

FIG. 1 shows a medical device embodying the present invention with a urine drainage bag attached and urine being transferred to the drainage bag;

FIG. 2 shows the medical device of FIG. 1 while an irrigation solution is introduced into the bladder of a female patient with the urine drainage bag attached;

FIG. 3 shows the device of FIG. 1 while an irrigation solution is introduced into the bladder of a female patient with the urine drainage bag detached;

FIG. 4 shows the device of FIG. 2 wherein the catheter is a leakage resistant insert;

FIG. 5 shows the medical device of FIG. 2 having a light source and a mirror attached to a syringe serving as the grip;

FIG. 6 shows the medical device of FIG. 4 having a light source and a mirror attached to a syringe by a pliant arm;

FIG. 7 shows the medical device of FIG. 4 having a camera attached to the syringe serving as the grip;

FIG. 8 shows the medical device of FIG. 4 having a pair of cameras attached to the grip;

FIG. 9 shows the top view of an embodiment of this invention provided with a removable visual aid holder and a mirror;

FIG. 10 shows the top view of an embodiment of this invention provided with a removable visual aid holder with a camera; and

FIG. 11 shows a top view of an embodiment of this invention provided with a removable visual aid holder with a pair of cameras.

### DESCRIPTION OF PREFERRED EMBODIMENTS

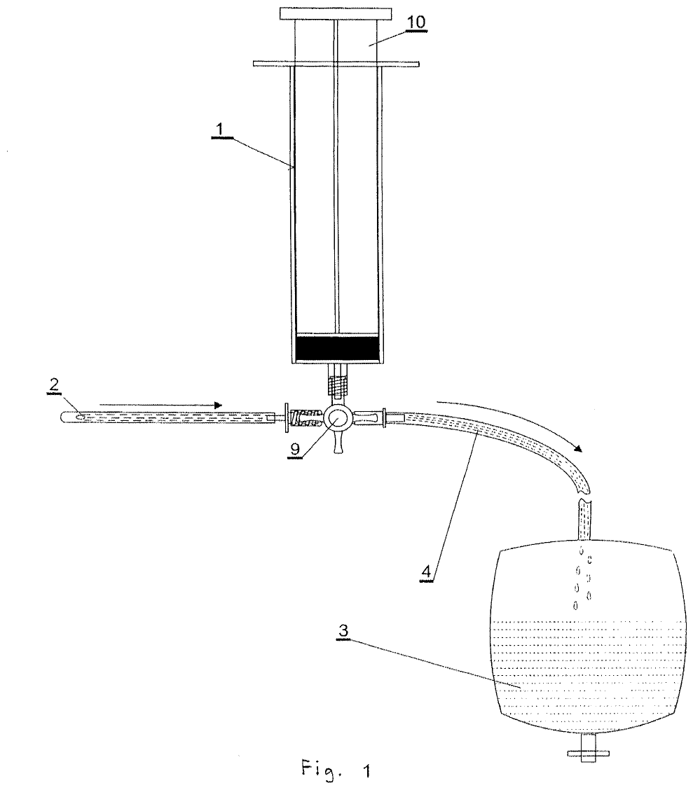

Referring to FIG. 1 female catheter 2 of the medical device is shown connected to three-way valve 9 which in turn is operably connected to syringe 1 provided with piston 10 and to urine drainage bag 3. Three-way valve 9 is positioned for direct flow from catheter 2 to urine drainage bag 3, thereby providing a closed system. Syringe 1 also connected to three-way valve 9, serves as a rigid grip for the medical device. In this particular embodiment, the syringe can be replaced by a solid elongated member, if desired, to serve as the grip or handle. The three-way valve can be provided with Luer-Lock connectors, if desired.

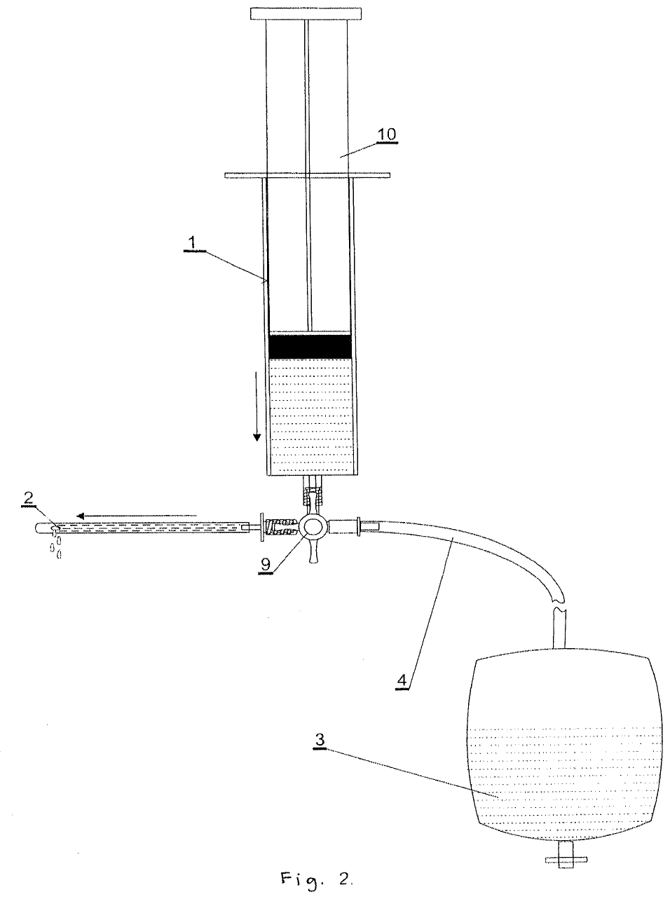

FIG. 2 is similar to FIG. 1 but shows three-way valve 9 positioned for liquid flow from syringe 1 to female catheter 2 and urine drainage bag 3 isolated.

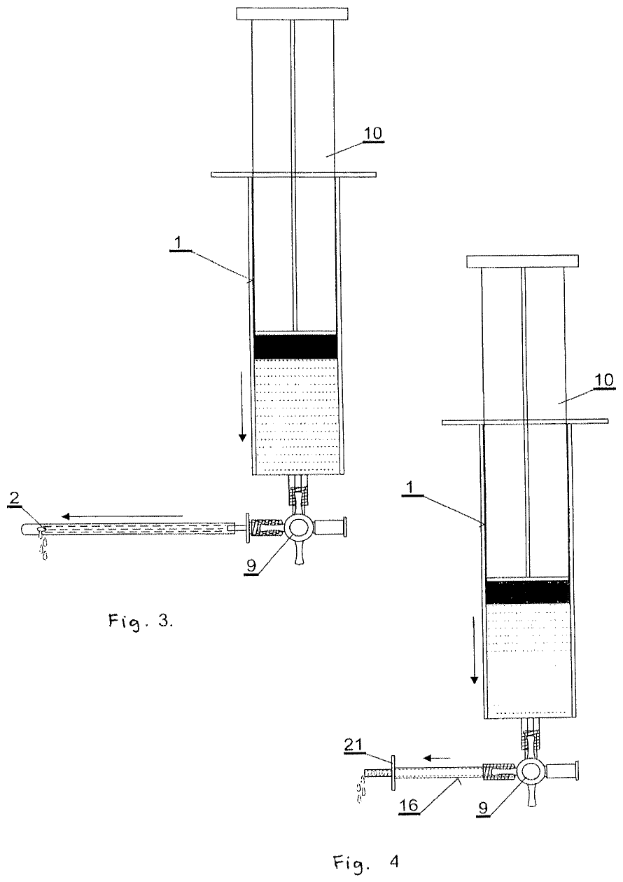

FIG. 3 shows syringe 1 operably connected to catheter 2 for liquid flow from syringe 1 to female catheter 2. Urine drainage bag 3 is disconnected from three-way valve 9.

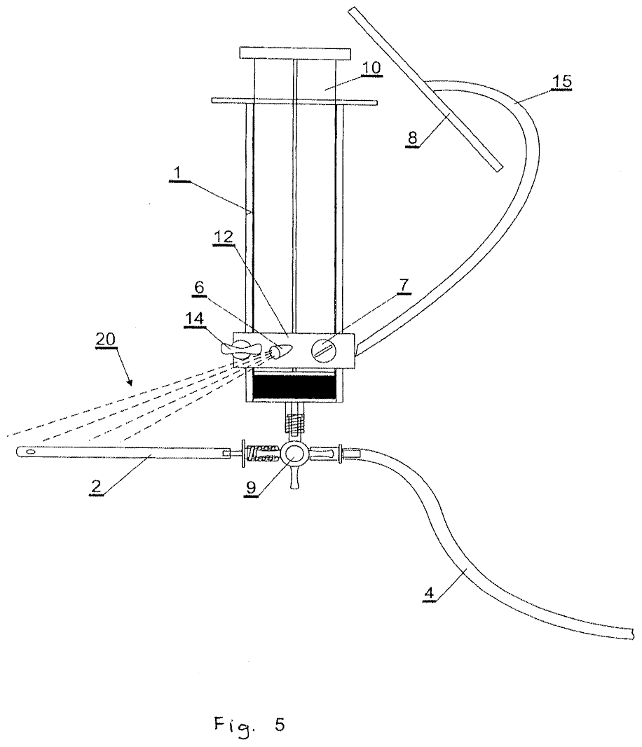

FIG. 4 shows syringe 1 operable connected via from three-way valve 9 to leakage resistant medicament instillation catheter 16 provided with sealing collar 21.

FIG. 5 shows syringe 1 with three-way valve 9 positioned for direct flow between female catheter 2 and urine drainage bag tube 4. Holder 12 is removably attached to syringe 1 by clamp 14 and carries adjustable light source 6, preferably LED diodes emitting beam 20, together with associated power source 7 for the light source. In addition, holder 12 carries mirror 8 which is attached to holder 12 by pliant arm 15 that permits positioning of mirror 8 by user for optimum view. Three-way valve 9 is in position for liquid flow from catheter 2 to urine drainage bag tube 4. Light source 6 is positioned to illuminate the female catheter insertion site. A switch (not shown) between light source 6 and power source 7 controls the light source. Preferably, the switch is waterproof. Mirror 8 is connected to pliant arm 15 by a ball joint. The size of mirror 8 can vary, but usually the mirror has a diameter of about 100 millimeters. Length of pliant arm 15 is selected so that mirror 8 can be positioned at a distance of about 100-200 millimeters from a urinary meatus when in use.

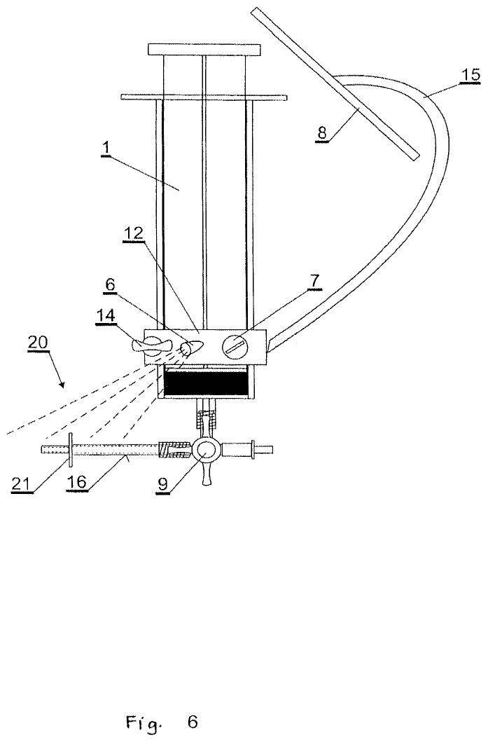

FIG. 6 shows three-way valve 9 operably connected to syringe 1 and medicament instillation catheter 16. In this particular embodiment, syringe 1 serves only as a grip or handle that facilitates catheter insertion.

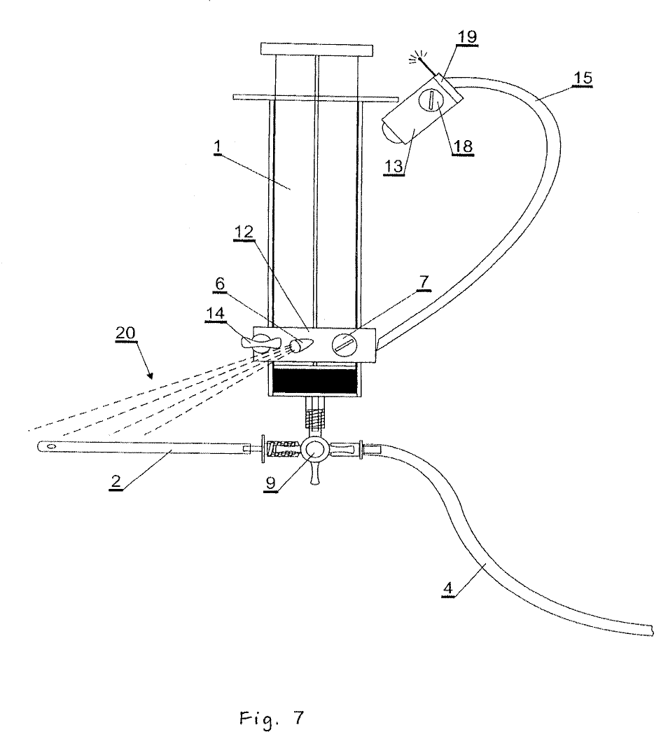

FIG. 7 shows three-way valve 9 operably connected to catheter 2 and to tube 4, and attached to syringe 1 which again serves only as the grip. Camera 13 with associated power source 18 and wireless transmitter 19 is attached to holder 12 by means of pliant arm 15 instead of a mirror. Wireless transmitter 19 can transmit an output of camera 13 to a suitable receiving device such as a smartphone, tablet, PC monitor, video monitor, and the like.

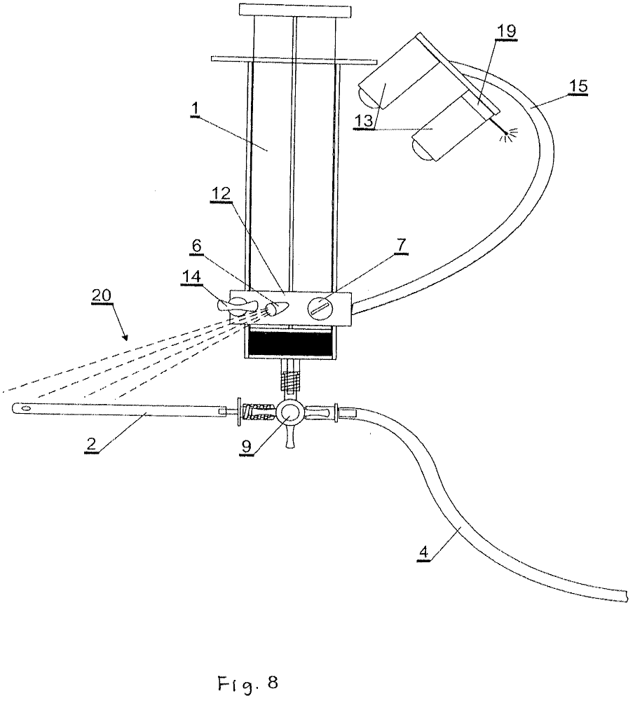

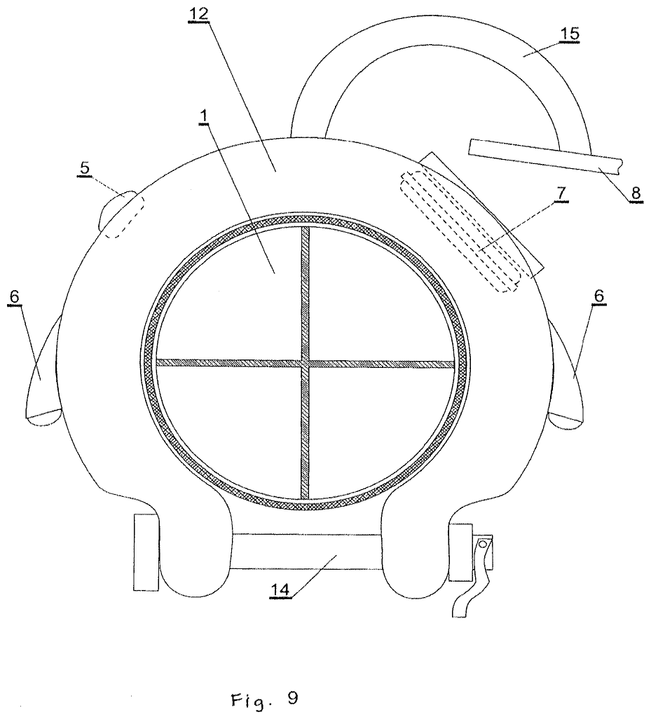

FIG. 8 shows a medical device similar to that shown in FIG. 7 but provided with a pair of cameras. In this manner, a three-dimensional picture of the insertion site can be conveyed to a smartphone, tablet, PC monitor, video monitor, and the like.

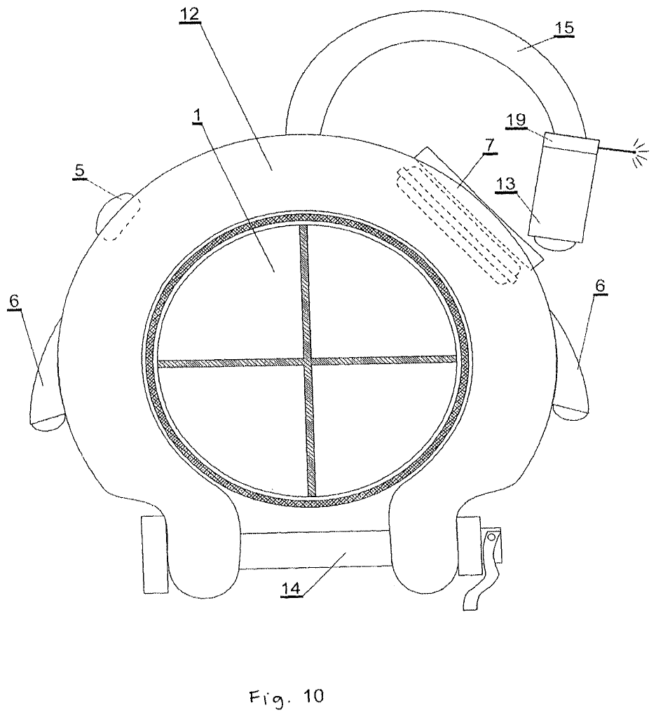

FIG. 9 shows top view of a medical device provided with dual light sources 6 that are energizable by waterproof microswitch 5 provided on holder 12 which also carries pliant arm 15 with mirror 8 at the distal end thereof, as well as power source 7 for dual light sources 6. Clamp 14 has a horseshoe-like configuration and is flexible.

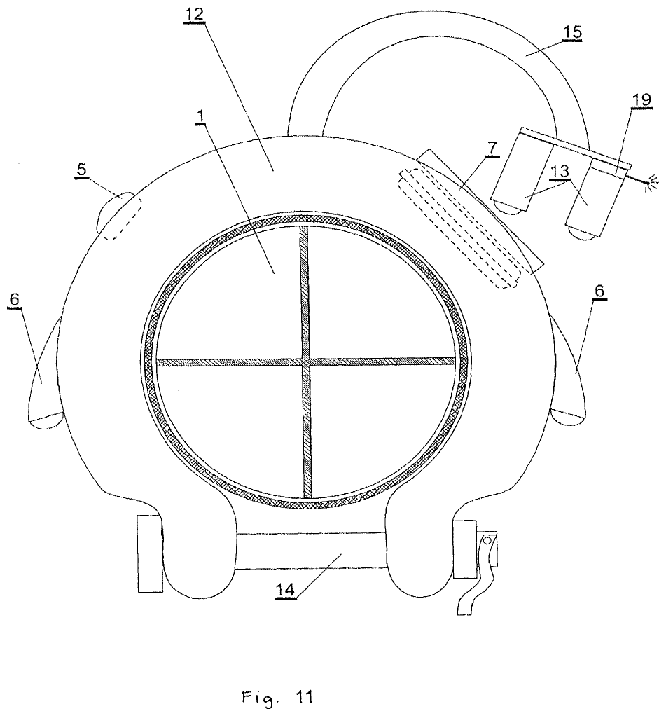

FIG. 10 shows a top view of a medical device similar to that shown in FIG. 9 but provided with a camera 13 and wireless transmitter 19 at the distal end of pliant arm 15. Microswitch 5 can also be utilized to switch on camera 13 as well as wireless transmitter 19.

FIG. 11 likewise is similar to FIG. 9 but is provided with a pair of cameras 13 at the distal end of pliant arm 15 operably associated with wireless transmitter 19.

To effect bladder drainage by self-catheterization, a commercially available, single use, female catheter having a length of about 150 millimeters to about 250 millimeters can be used. Preferred female catheters have a length of about 200 millimeters. The drainage bag 3 is connected to female catheter 2 through tube 4 of urine drainage bag 3 with the conic end. The combination of female catheter 2 and drainage bag 3 makes the intermittent clean self-catheterization in a closed system possible. Using this closed system regularly, (self-catheterization 2-3 times per day) only the single use bladder catheter needs to be changed to a new sterile catheter. The light source and the mirror or the camera fixed to the handle can be used advantageously for the quick and simple location of the external urethral orifice.

The device shown in FIG. 2 can be used by patients with urination problems who need medication in addition to drainage. For medication, syringe 1 containing a medicament solution is connected to three-way valve 9. The medicament solution can be introduced into the bladder from syringe 1 by proper setting of three-way valve 9. Before the instillation, urine drainage should be performed through tube 4 of urine drainage bag 3 into drainage bag 3.

The device shown in FIGS. 3 and 4 can be used by patients who need medication only. In such cases, urine drainage bag 3 is not needed. As shown in FIG. 4, a relatively longer catheter can be replaced by a shorter leakage resistant catheter 16. Sealing collar 21 of the shorter catheter 16 assists leakage-resistant introduction of the medicament solution.

In general, to use the present medical device, holder 12 is attached to grip 1. Grip 1 is connected to catheter 2 and urine drainage bag 3. Light source 6 on holder 12 is switched on by waterproof microswitch 5. Mirror 8 is adjusted to illuminate the target region. In case a camera 13 is used, the camera is switched on and directed to the target region. Necessarily, the devices for visualizing the pictures of camera 13 should be switched on as well.

After the target region has been illuminated, catheter 2 or leakage resistant catheter 16 can be guided to the target region for insertion into the urinary meatus. The insertion position of adapter 16 is assured by sealing collar 21.

Depending on the type of patent, use of the medical device is initiated in a specific manner. Concerning the first type of patient, where the objective is solely bladder drainage, syringe or handle 1 is connected to catheter 2 either through the three-way valve 9 or to tube 4 of urine drainage bag 3. In another type of patient, where there is no drainage needed, either catheter 2 or leakage resistant catheter 16 to be inserted into the urinary meatus is connected to the appropriate outlet of three-way valve 9.

The advantage of this invention is that there is no need to transport the patient regularly to a healthcare center or clinic to change the catheter. As a result, there is a much lower risk of developing chronic bacterial inflammation, there is no permanent irritation, no sensation of a foreign body, and no pain in the urethra. The complications of inflammation are less frequent and milder. The regular treatment of the is not subject to burdens such as consulting hours of the doctor, the need for home assistance, ambulance transport etc. Depending on the needs of the patient, the treatment can be customized.

Because of the construction of the device, the device can be operated with one hand only, so the other hand can be used to explore the vulva properly. The urinary meatus (external urethral orifice) can be easily located and sterilized.

By changing or removal of individual components of the medical device, the needs of all types of patients can be fulfilled. The use of the medical device can be learned in a relatively short time. A wide spectrum of needs is covered.

The use of the present medical device supports patient healthcare, and improves quality of life and life expectancy of patients who have serious conditions or are disabled. A significant reduction in the risk of complications due to catheterizations is possible. Learning to handle the device is relatively easy which assures independency for the patients from health care professionals, and by customizing the treatments according to the needs of the individual patient, an improvement in therapeutic results can be expected.

The foregoing description and the drawings are intended to be illustrative of the present medical device, and are not to be taken as limiting. Still other variants and arrangements of the individual parts within the spirit and scope of this invention are possible and will readily present themselves to those skilled in the art.

1. A medical device suitable for self-catheterization by a female patient which comprises
a female catheter;
a three-way valve operably associated with the female catheter;
a rigid grip attached to the three-way valve; and
at least one visual aid mounted to the rigid grip.

2. The medical device in accordance with claim 1 wherein the rigid grip is a syringe operably connected to the three-way valve.

3. The medical device in accordance with claim 1 wherein the visual aid is removably attached to the grip.

4. The medical device in accordance with claim 1 wherein the rigid grip is a syringe operably connected to the three-way valve and the valve and the visual aid is removably attached to the syringe.

5. The medical device in accordance with claim 1 wherein the visual aid is a mirror.

6. The medical device in accordance with claim 1 wherein the visual aid is a camera.

7. The medical device in accordance with claim 1 wherein the visual aid is a mirror connected to the rigid grip by a pliant arm.

8. The medical device in accordance with claim 1 wherein the visual aid is a light source.
